SEQUENCE LISTING (2 sequences)

SEQ ID NO: 1 - DNA, 19 bp, Artificial Sequence
  Description of Artificial Sequence Synthetic primer
ggtatcccatcaaggcccc

SEQ ID NO: 2 - DNA, 20 bp, Artificial Sequence
  Description of Artificial Sequence Synthetic primer
tttccctgtggtgtagggca

(19) United States
(12) Patent Application Publication
(10) Pub. No.: US 2011/0045477 A1
(43) Pub. Date: Feb. 24, 2011

(54) HUMAN SKIN EXPLANT CULTURE SYSTEM AND USE THEREFOR

(76) Inventors: Nannan Chen, Princeton, NJ (US); Yaping Hu, Highland Park, NJ (US); Connie Baozhen Lin, Belle Mead, NJ (US); Apostolos Pappas, Hillsborough, NJ (US); Miri Seiberg, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/858,471

(22) Filed: Aug. 18, 2010

Related U.S. Application Data
(60) Provisional application No. 61/235,923, filed on Aug. 21, 2009.

(51) Int. Cl. C12Q 1/68; C12N 5/071; G01N 33/566; C12Q 1/02

(52) U.S. Cl. 435/6; 435/371; 435/7.21; 435/29

(57) ABSTRACT

The present invention features a human skin explant culture system and uses thereof.

### CROSS REFERENCE TO RELATED APPLICATIONS

This application claims priority of U.S. Provisional Application 61/235,923 filed Aug. 21, 2009. The complete disclosure of the aforementioned related U.S. patent application is hereby incorporated herein by reference for all purposes.

### FIELD OF THE INVENTION

The present invention relates to a human skin explant culture system and use of the system for testing the effects of compositions on the metabolic activity of the skin.

### BACKGROUND OF THE INVENTION

In vitro model systems have always been a vital component of both basic and applied research. The development of such model systems for the skin is of increasing priority, due to the recent European Community regulation that bans the use of animal testing for cosmetic ingredients.

Human skin explants have been studied in culture for more than 50 years, mainly for epidermal biology and for epidermal cancer research. However, these model systems center on epidermal activity, and fail to reproduce dermal and adipose layer metabolic activity and tissue architecture.

A paper, “Repair of UVA-Induced Elastic Fiber and Collagen Damage by 0.05% Retinaldehyde Cream in an ex vivo Human Skin Model” by S. Boisnic et al. given at New Concepts for Topical Use of Natural Retinoids, Retinaldehyde in Perspective, Proceedings of a Satellite Symposium held at the 7th EADV Meeting, Oct. 7, 1998, Nice, France (Editors: J.-H. Saurat, Geneva, Switzerland; A. Vahlquist, Uppsala, Sweden), discusses the effects of retinaldehyde on collagen in skin explant cultures.

The authors of a recent publication, “Effect of Green Coffea Arabica L. Seed Oil on Extracellular Matrix Components and Water-channel Expression in in vitro and ex vivo Human Skin Models” (Journal of Cosmetic Dermatology, 2009 March; 8(1):56-62, Velazquez Pereda Mdel C et al.) were not able to show effects in explant cultures. Therefore, they used histological sections of human skin incubated with their test agents and immuno-stained to document stimulation in the synthesis of collagen, elastin, and other extracellular matrix components.

Despite the teachings of these papers, there is a continuing need for a human skin explant model system that best represents the physiological complexity, the metabolic activity, and the structural integrity of all skin compartments.

There is also a need to increase the surface area of cultured biopsies, in order to enable studies of topical applications of agents and compositions. The standard biopsy size of 4 mm in diameter does not support such studies. “A Human Full-Skin Culture System for Interventional Studies” (Eplast.2009; 9:e5. Published online 2009 Jan. 9, by Lars Steinstraesser et al.), describes the culture of larger skin biopsies and documents the preservation of histological properties of the skin explants for 4 weeks. However, study of a transgene expression pattern of this explant system was found not to mimic the in vivo observed metabolic activity.

There is a continuing need for a human skin explant model system that best represents the physiological complexity, the metabolic activity, and the structural integrity of all skin compartments, and has a sufficient surface area to enable topical treatment with test agents and compositions.

### SUMMARY OF THE INVENTION

The present invention is directed to a human skin explant culture system comprising a human skin biopsy having a diameter up to about 25 mm in a medium comprising: about 40% to about 60% by volume of Dulbecco's modified Eagle's medium; about 40% to about 60% by volume of F-12 nutrient mixture; about 0.5% to about 5% by weight of fetal bovine serum; 1 to 20 μg/ml of insulin; 1 to 20 ng/ml of hydrocortisone, 1 to 20 ng/ml of epidermal growth factor; and 1× antibiotic antimycotic.

The present invention also provides a method for determining an effect of a composition for topical application to skin comprising: incubating a skin biopsy having a diameter up to about 25 mm in a medium comprising: about 40% to about 60% by volume of Dulbecco's modified Eagle's medium; about 40% to about 60% by volume of F-12 nutrient mixture; about 0.5% to about 5% by weight of fetal bovine serum; 1 to 20 μg/ml of insulin; 1 to 20 ng/ml of hydrocortisone, 1 to 20 ng/ml of epidermal growth factor; and 1× antibiotic antimycotic to create a skin explant culture system; topically applying the composition onto the skin biopsy; and analyzing a biological response of the skin biopsy to the composition. In another embodiment, the composition or test agent is applied into the culture media described above, in order to separate the biological effect of the composition on the different skin compartments from the effect of the topical delivery.

### DETAILED DESCRIPTION OF THE INVENTION

The culture system of the present invention is useful for extending the viability of skin explants and for enabling metabolic activity of all layers of the skin explants, which enables the study of effects of topically applied compositions.

Commonly, skin explants are used as skin biopsies with a diameter of 2-4 mm in size, since larger explants undergo necrosis at the center of the tissue under standard culture conditions. However, skin explants with such a small size are not suitable for topical application. The optimal media to support the integrity of larger human skin explants, which enables the evaluation of topically-applied dermatological actives, has now been identified.

The culture system comprises a medium containing Dulbecco's modified Eagle's medium (“DMEM”) with a high sucrose content. The Dulbecco's modified Eagle's medium may be obtained, for example from Invitrogen Corporation, Carlsbad, Calif., USA as Dulbecco's Modified Eagle Medium (D-MEM) (1×), liquid (high glucose)/cat#: 11965.

The amount of DMEM may range from about 40 to about 60 percent by volume, for example, about 50 percent by volume, of the medium.

The medium also contains F-12 nutrient mixture (“F-12”). The F-12 nutrient mixture may be obtained, for example from Invitrogen Corporation, Carlsbad, Calif., USA as F-12 Nutrient Mixture (Ham) (1×), liquid12/cat#: 11765.

The amount of F-12 nutrient mixture may range from about 40 to about 60 percent by volume, for example, about 50 percent by volume, of the medium.

The medium further includes bovine serum, for example fetal bovine serum. The bovine serum may be obtained, for example from Invitrogen Corporation (Carlsbad, Calif., USA) as Fetal Bovine Serum, Certified, Heat-Inactivated/cat#: 10082-139.

The amount of bovine serum may range from about 0.5 to about 5 percent by weight, for example, about 2 percent by weight, of the medium.

The medium is supplemented with insulin, hydrocortisone, epidermal growth factor (“EGF”), and antibiotic antimycotic (“ABAM”).

The amount of insulin may range from 1 to 20 μg/ml, for example 10 μg/ml. The insulin may be obtained, for example from Sigma (St. Louis, Mo., USA) as insulin solution human/cat#: I9278.

The amount of hydrocortisone may range from 1 to 20 ng/ml, for example 10 ng/ml. The hydrocortisone may be obtained, for example from Sigma (St. Louis, Mo., USA) as hydrocortisone powder, γ-irradiated/cat#: H0135).

The amount of epidermal growth factor may range from 1 to 20 ng/ml, for example 10 ng/ml. The epidermal growth factor may be obtained, for example from Invitrogen Corporation (Carlsbad, Calif., USA) as Recombinant Human Epidermal Growth Factor (EGF)/cat#: PHG0311.

The amount of antibiotic antimycotic is 1×. The antibiotic antimycotic may be obtained, for example from Invitrogen Corporation (Carlsbad, Calif., USA) as Antibiotic-Antimycotic (100×), liquid/Cat. No. 15240-062.

Human skin explants of up to about 25 mm in diameter, for example from about 2 to about 25 mm, or about 4 to about 25 mm, or in certain embodiments about 12 mm in diameter, are placed in the medium. The medium should be leveled with the height of the explants.

In one embodiment, the explants are incubated at about 32° to about 37° C., for example about 32° C. It has been unexpectedly found that reducing the culturing temperature from 37° C. (standard temperature) to about 32° C. enables longer survival and better integrity and metabolic activity of the explants. It has been also unexpectedly found that reducing the culturing temperature from 37° C. (standard temperature) to about 32° C. for the first 24 hours of culturing, and then incubating the explants at 37° C., also enables longer survival and better integrity and metabolic activity of the explants.

In another embodiment, the amount of fetal bovine serum is reduced from 5% to 2%. This also enables longer survival, better tissue integrity and better metabolic activity of the cultured explants.

In another embodiment, the explants are incubated in a standard humidified atmosphere containing 5% by volume CO₂.

The culture medium is refreshed daily. That is, the media and nutrients are removed and replaced.

The culture medium used in the present invention enables tissue viability. As used herein, “enabling tissue viability” means the enabling of tissue survival in culture and the prevention of tissue damage that leads to cell and tissue death, such as the prevention of tissue necrosis.

Tissue viability may be demonstrated by histological analysis of histologically-stained tissue sections, and the demonstration of intact and normal tissue architecture.

Tissue viability may also be measured by the analysis of gene expression of genes known to be essential to cell viability. Such genes include, but are not limited to, a group of genes defined as “housekeeping genes. Housekeeping genes are typically constitutive genes that are transcribed at a relatively constant level across many or all known conditions. The products of the housekeeping genes are typically required for the maintenance of the cell. It is generally assumed that the expression of housekeeping genes is not affected by topical treatments of non-toxic agents. Examples of housekeeping genes include, but are not limited to actin, GAPDH, 18S RNA and ubiquitin. Tissue viability may also be measured by any means known to those skilled in the art.

The culture system of the present invention enables the study of effects of compositions for topical application to the skin. The molecular, cellular and/or physiological responses of the skin explants to the tested composition may be measured. The skin explants may be analyzed through histology, molecular analyses, biomarker analysis, and the like.

Specifically, the current invention enables higher level of, and more resemblance to the metabolic activity of skin in vivo, in all compartments of the skin explant. The metabolic activity of the three compartments of the skin may be measured using explants cultured according to the invention. As used herein, “metabolic activity” means the active gene expression or the synthesis of gene products or the activity of proteins such as enzymes, and the creation of end-products, which are specialized for these tissue compartments and are not only essential for tissue viability or survival.

In one embodiment, the metabolic activity of an explant cultured according to the invention is analyzed by gene expression of tissue-specific genes.

For the epidermal compartment of the skin, such genes include, but are not limited to, keratinocyte-expressed genes such as specific keratins such as keratins 5, 14, 1 and 10, PAR-2, or KGFR, and melanocyte specific genes such as tyrosinase, TRP-1 and TRP-2 and other melanogenic genes.

For the dermal compartment of the skin, such genes include, but are not limited to, elastin, elastin-accessory proteins such as Fibrilin-1 and fibulin-5, and collagens such as collagen1α1 and collagen 4.

For the adipose layer of the skin, such genes include, but are not limited to, lipogenic genes, such as PPAR-γ, leptin, GLUT4, FABP4, AdPLA₂ and Pref-1, and lipolytic genes, such as PPAR-α, acyl-CoA dehydrogenase, phosphodiesterase, CPT carnitylpalmitoyltransferase and GPR81.

In another embodiment of this invention, the metabolic activity of the dermal layer of the skin is analyzed by histological or immunohistochemical staining of tissue sections of an explant cultured according to the invention. Examples of such stainings include, but are not limited to, Luna elastin staining that documents enhanced elastin fiber network, or pre-collagen immunohistochemical staining that documents new collagen synthesis.

In yet another embodiment, the metabolic activity of the adipose layer of an explants according to the invention is measured by analysis of molecules involved in lipid metabolism that are secreted into the culture media of these explants. Examples of such molecules include, but are not limited to, secreted proteins such as leptin, and the secretion of lipid molecules such as glycerol and non-esterified fatty acids.

Unless defined otherwise, all technical and scientific terms used herein have the same meaning as commonly understood by one of ordinary skill in the art to which the invention belongs. Whenever used, any percentage is weight by weight (w/w) unless otherwise indicated.

Examples of the present invention are described below. The invention should not be construed to be limited to the details thereof.

### Example 1

Human abdominal skins were obtained with informed consent from healthy individuals undergoing plastic surgery. Patient identities were not disclosed to preserve confidentiality, in compliance with US HIPAA regulations. Punch biopsies (4 and 12 mm in diameter) were first disinfected at room temperature for 30 min with DMEM supplemented with Pen/Strep (200 unit/ml Pen, 200 μg/ml strep), fungizone (5 ug/ml), and gentamycine (20 ug/ml), all obtained from Invitrogen, Carlsbad Calif. The explants were placed in the different media listed in Table 1, supplemented with antibiotics and a cocktail of growth factors listed in Table 2, and placed in a humidified chamber at 37° C. in a 5% CO₂ atmosphere. Media were refreshed daily.

Media A-G were comparative. Medium 1 was according to the invention.

TABLE 1
Media Tested (unless otherwise indicated, all
materials were purchased from Invitrogen)

| | |
|---|---|
| Medium A | BioEc media (Laboratoire BIO-EC, Clamart, France) 5% |
| | Fetal Bovine Serum (FBS), 50 μg/ml bovine pituitary |
| | extract (BPE),1x antibiotic-antimycotic (ABAM) |
| Medium 1 | 50% Dulbecco's Modified Eagle's Medium (DMEM) + 50% |
| | F-12 Nutrient Mixture (F12), 5% FBS, with supplement listed |
| | in Table 2, except BPE |
| Medium B | Williams' Media E, 5% FBS, with supplement listed in |
| | Table 2 |
| Medium C | KBM-2, with vendor's supplement (Lonza, Walkersville, |
| | MD) |
| Medium D | KBM-2, 5% FBS, 50 μg/ml BPE, 1x ABAM |
| Medium E | 50% DMEM + 50% KBM-2, 5% FBS, with supplement listed |
| | in Table 2 |
| Medium F | 50% DMEM + 50% F-12, 5% FBS, with supplement listed |
| | in Table 2 |
| Medium G | 50% DMEM + 50% F-12, 5% FBS, with supplement listed |
| | in Table 2, except BPE |

TABLE 2
Media Supplements

insulin (10 μg/ml)
hydrocortisone (10 ng/ml)
EGF (10 ng/ml)
with and without BPE (50 μg/ml) as indicated.
1x ABAM

Skin explants were harvested after defined time periods, fixed overnight in 10% formalin (Richard-Allan scientific, Kalamazoo, Mich.), and then stored in 70% ethanol. The samples were then embedded into paraffin blocks and sectioned (5 μm), and processed for hematoxylin and eosin (H&E) staining using standard procedures. Images of the stained sections were obtained using Leica microscope (Leitz DM1L, Leica, Allendale, N.J.) and a QiCAM camera (QIMAGING, Surrey, BC, Canada). At least 12 images from each tested condition were graded, by expert graders, for tissue integrity, with a focus on epidermal cells integrity and dermal collagen degradation.

No necrosis was identified up to 12 days for the explants cultured in Medium 1 for either the 4 mm (standard size) or 12 mm (large size) explants. Minimal or no vacuolated cells were observed in the epidermis, and no extracellular matrix degradation was detected in the dermis up to day 12 of culture. In contrast, necrosis, dermal matrix degradation and vacuolated epidermal cells were observed at 12 days of culture or at earlier time points using Media A-G.

Table 3 provides the data from a representative experiment comparing Medium 1 and Medium A. Similar studies with the other comparative media listed in Table 1 and the supplements listed in Table 2 confirmed the superiority of Medium 1. Each data point presented in Table 3 represents 3 large biopsies (12 mm). The grading scale for these studies ranged from 1-5, with 5 having best tissue integrity. For each study, the integrity of the tissue immediately prior to culturing (named “pre-culture” here) was defined as 5.

TABLE 3

| | |
|---|---|
| Medium A | Medium 1 |

| | epidermal | dermal | epidermal | dermal |
|---|---|---|---|---|
| Pre-culture | 5 | 5 | 5 | 5 |
| Day 3 | 4.5 | 4.5 | 4.5 | 4.5 |
| Day 6 | 3 | 3 | 4 | 4 |
| Day 12* | 0 | 2 | 3 | 3 |

*Day 12 studies were performed in separate experiments.

The data in Table 3 demonstrates that culture Medium 1 (containing 5% FBS) according to the invention is superior to the comparative Medium A in maintaining viable skin organ culture with a longer survival time.

### Example 2

Human skin explant cultures (12 mm) were established in Medium 1 containing 2% FBS as described in Example 1. Explant cultures were either incubated at 37° C. or at 32° C., in a 5% CO₂ atmosphere. Media were refreshed daily. After predefined time periods from starting of the experiment, skin explants were harvested for histological staining and were evaluated as described in Example 1. Table 4 presents data from a representative experiment comparing 37° C. to 32° C. Each data point represents 3 biopsies.

TABLE 4

| | |
|---|---|
| Days | Temperature |

| post culture | 37° C. | 32° C. |
|---|---|---|
| Pre-culture | 5 | 5 |
| Day 5 | 4.5 | 4.5 |
| Day 7 | 4.5 | 4.5 |
| Day 9 | 4.5 | 4.5 |
| Day 12 | 3.5 | 4.5 |

The data in Table 4 demonstrates that skin explant cultures incubated for 12 days at low temperature (32° C.) have superior metabolic activity compared to explants fro the same donor skin incubated at standard temperature (37° C.). In addition, longer survival of skin explant cultures is achieved using the culture medium of this invention with lower levels of serum (2%) (as documented in Table 3, Medium 1 with 5% FBS and in Table 4, Medium 1 with 2% serum). Additionally, skin explants incubated at lower temperature (32° C.) for the first 24 hours and then switched to standard temperature (37° C.) have longer survival than explants continuously incubated at 37° C.

### Example 3

Since viable tissue explants can be either metabolically active, or have only low metabolic activity, or could be dormant, and since it is desired to use metabolically active skin organ culture for the evaluation of dermatological agents, we tested culturing under the optimized culture conditions of the invention for the ability to support metabolic activity in culture.

Skin explant cultures were established as described in Example 1, using Medium 1. Explants were incubated at 37° C. in a 5% CO₂ atmosphere. Skin explants either remained untreated or were treated, in Medium 1, with TGF-β, an agent known to increase elastin production. Media were refreshed daily. After predetermined time periods, the skin explants were harvested, and processed for histological, immunohistochemical, and gene expression evaluation as follows.
1. LUNA staining was used to document elastin fibers histologically, as described in Kligman, Am. J. of Dermatopathology, 3(2): 199-201, 1981. The grading of elastin fiber quality ranged from 1-5, when 5 represented best fiber quality.
2. Ki67 was used as a marker for cell proliferation, using immunohistochemistry techniques with a rabbit monoclonal antibody for Ki67, purchased from Thermo Scientific (Pittsburgh, Pa.). Immunohistochemistry staining was performed according to manufacturer's instructions. The grading of Ki67 ranged from 1-5, when 5 represented normal Ki67 level in the skin sample prior to culture.
3. Levels of elastin gene expression (mRNA levels) were evaluated by QPCR. Total RNA was extracted from skin explants using Trizol (Invitrogen, Carlsbad Calif.) according to manufacturer's instructions. RNA was then converted to cDNA using Superscript® III reverse transcriptase (Invitrogen, Carlsbad, Calif.), and QPCR analyses were performed using a 7300 Realtime PCR system (Applied Biosystems, Foster City, Calif.).
Elastin primers: forward: GGTATCCCATCAAGGCCCC reverse: TTTCCCTGTGGTGTAGGGCA. QPCR reaction, in a 20 μl volume, contained 10 μl QPCR master mix (Applied Biosystems, Foster City, Calif.), 1.5 μl of either forward or reverse primer (5 μM), 5 μl of cDNA, and 2 μl of H₂O). Untreated control was normalized to 100%.

Table 5 provides data from a representative experiment.

TABLE 5

| | Day 5 | | Day 7 | |
|---|---|---|---|---|
| | Untreated | TGF-β | Untreated | TGF-β |
| Parameters examined | Control | (20 ng/ml) | control | (20 ng/ml) |
| Ki67 | 4 | 4 | 4 | 4 |
| Proliferation | | | | |
| marker | | | | |
| LUNA staining | 3 | 3 | 3.5 | 4 |
| Elastin fibers | | | | |
| Elastin mRNA | 100% | 369% | 243% | 423% |
| (QPCR) | | | | |

The data in Table 5 demonstrates the metabolic activity of the dermal compartment of skin explants cultured according to the invention. The positive response of the tissues to TGF-β further confirms their metabolic activity, as elastin is induced under the optimized culture conditions in response to TGF-β.

### Example 4

PPAR-γ activation is an essential regulator of adipocyte proliferation, differentiation, maintenance, and survival (Anghel, et al, J. of Biol. Chem., 282(41), 29946-57, 2007). Rosiglitazone, a PPAR-γ agonist, induces adipocyte differentiation (Patel et al., Diabetes. 52(1):43-50, 2003). On the other hand, conjugated linoleic acid attenuates lipogenesis and induces fatty acid oxidation (Evans et al., J Nutr.; 132(3):450-5, 2002; Brown et al., J Nutr.; 131(9):2316-21, 2001).

To examine the metabolic activity of the subcutaneous adipose layer, explant cultures were established as described in Example 1, using optimized media with 5% serum. After overnight incubation, skin explants remained untreated or were treated with 20 μM of rosiglitazone or with 50 μM of conjugated linoleic acid in the media, for the evaluation of their effect on lipogenesis and lipolysis of subcutaneous adipose layer. Explants were incubated at 37° C. in a 5% CO₂ atmosphere.

For assessing lipogenesis, skin explants were cultured in medium containing 20 μM of rosiglitazone and C-14 labeled acetate for 24 hours. Subcutaneous fat was then harvested, and the levels of triglyceride were determined by HPTLC as described in (Pappas et al., JID 118 (1) 164-171, 2002).

For evaluating lipolysis, skin explants were cultured in medium containing 50 μM of conjugated linoleic acid, and culture media were collected at indicated time points. The levels of glycerol released into the media were determined using the free glycerol reagent and kit (Sigma), which was used according to manufacture instruction.

The results of a representative study are shown in Table 6.

TABLE 6

| | Untreated | Rosiglitazone | Conjugated Linoleic |
|---|---|---|---|
| Parameters | control | (20 μM) | acid (50 μM) |
| Triglyceride synthesis | 100% | 240-270% | |
| at 24 hr (lipogenesis) | | | |
| Glyceral release | 100% | | 140% |
| at day 3 (lipolysis) | | | |

The data in Table 6 demonstrates the metabolic activity of the adipose layer of the skin explants cultured according to the invention. The positive response of the skin explants to both rosiglitazone (increase of triglycerides) and to conjugated linoleic acid (increase in glycerol release) documents a metabolically active adipose layer of the cultured skin explants.

What is claimed is:

1. A human skin explant culture system comprising a human skin biopsy having a diameter up to about 25 mm in a medium comprising:
about 40% to about 60% by volume of Dulbecco's modified Eagle's medium;
about 40% to about 60% by volume of F-12 nutrient mixture;
about 0.5% to about 5% by weight of fetal bovine serum;
1 to 20 μg/ml of insulin; 1 to 20 ng/ml of hydrocortisone, 1 to 20 ng/ml of epidermal growth factor; and 1× antibiotic antimycotic.

2. The culture system according to claim 1 comprising about 50% by volume of Dulbecco's modified Eagle's medium; about 50% by volume of F-12 nutrient mixture, and about 2% by weight of fetal bovine serum.

3. The culture system according to claim 1 comprising about 10 ng/ml of epidermal growth factor.

4. The culture system according to claim 1 incubated at about 32° to about 37° C.

5. The culture system according to claim 1 incubated at about 32° C.

6. The culture system according to claim 1 incubated at about 32° C. for 24 hrs and then incubated at about 37° C.

7. A method for determining an effect of a composition for topical application to skin comprising:
incubating a skin biopsy having a diameter up to about 25 mm in a medium comprising:
about 40% to about 60% by volume of Dulbecco's modified Eagle's medium;
about 40% to about 60% by volume of F-12 nutrient mixture;
about 0.5% to about 5% by weight of fetal bovine serum;
1 to 20 μg/ml of insulin; 1 to 20 ng/ml of hydrocortisone 1 to 20 ng/ml of epidermal growth factor; and 1× antibiotic antimycotic
to create a skin explant culture system;
topically applying the composition onto the skin biopsy; and
analyzing a biological response of the skin biopsy to the composition.

8. The method according to claim 7 wherein the culture system comprises about 10 ng/ml of epidermal growth factor.

9. The method according to claim 7 wherein the culture system is incubated at about 32° C.

10. The method according to claim 7 wherein the culture system is incubated at about 32° to about 37° C.

11. The method according to claim 7 wherein the culture system is incubated at about 32° C. for 24 hrs and then incubated at about 37° C.

12. The method according to claim 7 wherein the analyzing step is a dermal analysis comprising monitoring elastin fiber production through a process selected from the group consisting of LUNA staining; QPCR or transcription analyses of elastin or elastin accessory genes; protein detection of elastin or elastin accessory genes; histological staining; and immunohistochemical staining.

13. The method according to claim 7 wherein the analyzing step is a dermal analysis comprising monitoring collagen synthesis through a process selected from the group consisting of transcription of different collagen genes; protein detection of different collagen proteins; histological staining; and immunohistochemical staining.

14. The method according to claim 7 wherein the analyzing step is an analysis of the skin adipose layer comprising biochemical analysis for triglyceride levels post treatment with adipocyte differentiating agents to monitor lipogenesis.

15. The method according to claim 7 wherein the analyzing step is an analysis of the skin adipose layer comprising determination of free glycerol release after the use of a lipogenic agent to demonstrate lipolysis.

16. The method according to claim 7 wherein the analyzing step is an analysis of the skin adipose layer comprising monitoring leptin metabolism through a process selected from the group consisting of transcription of the leptin gene; protein detection of secreted leptin; and immunohistochemical staining.
